(19) United States
(12) Patent Application Publication
(10) Pub. No.: US 2009/0240281 A1
(43) Pub. Date: Sep. 24, 2009

(54) METHOD FOR PRODUCING DISPOSABLE TWEEZER TIPS FOR MICROSURGERY TWEEZERS AND MICROSURGERY TWEEZERS PROVIDED WITH SUCH TIPS

(76) Inventor: Jean-Marie Andre, Marseille (FR)

Correspondence Address: EGBERT LAW OFFICES, 412 MAIN STREET, 7TH FLOOR, HOUSTON, TX, 77002, US

(21) Appl. No.: 12/280,979

(22) PCT Filed: Feb. 27, 2007
(86) PCT No.: PCT/FR07/00354

§ 371 Date: Oct. 16, 2008

(30) Foreign Application Priority Data
Feb. 28, 2006 (FR) 0601766

(51) Int. Cl. A61F 9/007; A61B 17/50; B23P 17/00

(52) U.S. Cl. 606/210; 29/527.4

(57) ABSTRACT

The invention relates to a method for producing disposable tweezer tips for ophthalmological microsurgery tweezers for cataract surgery, especially those with a grip portion in a single piece of plastic and two arms interconnected by one of the ends thereof in order to form the proximal portion of the tweezers. There is also a tweezer portion having metal tips inserted into the distal ends of the arms during the molding of the grip part. The tweezer tips with the grip teeth are cut out and shaped on a cutting press provided with suitable cutting tools from a metal strip or plate having a tensile strength coefficient of between 350 and 550 Rm N/mm².

### CROSS-REFERENCE TO RELATED U.S. APPLICATIONS

Not applicable.

### STATEMENT REGARDING FEDERALLY SPONSORED RESEARCH OR DEVELOPMENT

Not applicable.

### NAMES OF PARTIES TO A JOINT RESEARCH AGREEMENT

Not applicable.

### REFERENCE TO AN APPENDIX SUBMITTED ON COMPACT DISC

Not applicable.

### BACKGROUND OF THE INVENTION

1. Field of the Invention

The present invention concerns a manufacturing method for disposable tweezer tips for ophthalmological microsurgery dedicated to cataract surgery. It pertains also to the tweezer tips or teeth resulting from the application of this method, and the microsurgery tweezers provided with such tips.

Generally speaking, the invention refers to a manufacturing method for tweezer tips for disposable microsurgery tweezers of the type featuring two arms joined to each other at one of their ends so as to form the proximal part of prehension of the tweezer and whose opposite ends are constituted by tweezer tips that are capable of being brought closer together in an elastic manner and which form the active part of said tweezer.

2. Description of Related Art Including Information Disclosed Under 37 CFR 1.97 and 37 CFR 1.98.

Operations of ophthalmological microsurgery, and in particular cataract operations which are performed under microscopic control, require the use of high precision tweezers, indeed of ultra precise tweezers, equipped with extremely fine tweezer tips. For example, such tweezer tips may present, in their distal part, a very reduced section of 0.25×0.25 mm and extremely fine prehensile micro-teeth of a length in the order of 0.1 mm.

This is a very specific category of high precision tweezers for which the specialist who has extensive experience in this type of microsurgery instrumentation is different from the specialist versed in the utilization or manufacture of other kinds of surgery tweezers.

Until now, the expert (ophthalmology surgeon) was convinced that such high precision instruments had to be made of a very hard material (see Afnor Standards NF S94-090, April 2001: Material for surgical instruments—martensitic stainless steels hardened by precipitation, austenitic and austeno-ferritic steels) in order to avoid any deformation of their tweezer tips and the blunting of their micro-teeth.

Today, under these conditions, these high precision instruments are most often made of hard and stainless metal, by skilled manual methods which make them very costly. These instruments generally made of martensitic steels, of austenitic steels or yet of austeno-ferritic steels (in particular of stainless 302 steel) known in professional circles as “surgical steels” on account of their good hardness characteristics and their bio-compatibility. Their high manufacturing cost is a major obstacle to offer these as “throw away” instruments, when their non-reuse would be desirable from a sanitary and economic standpoint. As a matter of fact, the obligatory sterilization and/or decontamination of reusable surgical instruments and their maintenance in an aseptic condition, between two successive utilizations, require substantial investments in qualified personnel, in equipment and working hours. On the other hand, these instruments become unusable as soon as their micro-teeth become a little bit deformed or blunted and no longer perform with the indispensable precision.

In order to bring down the cost of these instruments so as to make them disposable after one use, their manufacture has to be mechanized.

To achieve this objective a disposable surgical tweezer has already been proposed that is made entirely by molding under pressure of a thermoplastic material and presenting itself as a single monobloc piece.

The idea to produce ocular microsurgery tweezers in thermoformed plastic material may appear at first to be very attractive, because of the economical nature of this production method. Practitioners, however, have been able to notice that even if this material is suitable for the manufacture of the sleeves or proximal parts of the branches of the instruments, it does not display the indispensable physical characteristics of fineness and hardness inherent to metal, in the active distal part (tweezer tips of the branches) of said instruments to make them usable for microsurgery.

To remedy the aforementioned drawbacks of surgical instruments made entirely of metal or of surgical instruments made of nothing but plastic, certain ones of these instruments have already been proposed with a metallic active part that has been compound-filled by a plastic material injected under pressure, constituting the sleeve or the proximal prehension part of said instruments.

French Patent Document FR-03/15172 describes, for example, a surgical tweezer, in particular an ophthalmological microsurgery tweezer, consisting of a prehension part molded of a single piece of plastic and formed by two arms joined to each other at one of their ends to form the proximal part of the tweezer, and a tweezer part consisting of metallic tips or teeth inserted in the distal ends of the arms, during the moulding of said prehension part.

Thanks to such a manufacturing method, it is possible to produce surgical tweezers in a more economical way, making it possible to envisage reserving them for single use.

However, the cost of such composite surgical tweezers remains relatively high because the points are made of martensitic stainless steels, of austenitic steels or even of austeno-ferritic steels (see Afnor Standards NF S94-090 April 2001). The mechanical characteristics of which, in particular the hardness, are not compatible with any mechanized method allowing to obtain the required precision. These ultra-precise points are hence inevitably made one at a time, by artisan hands, which, of course, increases their cost.

### BRIEF SUMMARY OF THE INVENTION

One aim of the present invention is therefore the design of a method permitting for the first time complete mechanization of the production of the tweezer tips or teeth of the most precise, presently existing surgical tweezers, that is to say of tweezers for ocular microsurgery, this allowing therefore for the first time a mass production of these instruments thanks to which their production cost can be very substantially reduced.

More precisely, the invention aims to obtain single use microsurgery tweezers that must be disposable and non-reusable.

For this, the invention has led to researching a material other than the usual high hardness steels and featuring at the same time:
characteristics of corrosion resistance compatible with their utilization as disposable surgical instruments;
characteristics of bio-compatibility thanks to which the tweezer tips are usable during micro-surgical interventions;
characteristics of tenderness permitting the cutting of extremely fine and precise parts on a cutting press;
characteristics of rigidity or of hardness thanks to which the tweezer tips are capable of fulfilling their functions in a very reliable manner during the first operation of microsurgery; and
characteristics of tenderness which, because of the considerable fineness of the tweezer tips provided with prehension teeth of 0.1 mm length, result in the useful life of these to be limited, and that, although completely functional and available during the first intervention of microsurgery, these prehension micro-teeth become progressively dull, without losing any material, until they become non-existent and no longer functional at the end of the intervention of microsurgery. This characteristic prohibits a repeated reutilization, not intended by the manufacturer or the practitioner of these disposable tweezers.

A reduced cost makes it possible to produce disposable tweezers for microsurgery.

This research led to a solution which runs counter to the prevailing opinion among the specialists. According to which, the tweezer tips of microsurgery tweezers must absolutely be made of stainless so-called “surgical” steel, which is a specific stainless steel of great hardness (see Afnor Standards NF S94-090, April 2001: Material for surgical instruments—martensitic stainless steels hardened by precipitation, austenitic and austeno-ferritic steels).

According to the invention, the aforementioned objectives have been achieved thanks to a method according to which the tweezer tips of microsurgery tweezers are produced by high precision metallic cutting in a high precision die-cutting press. The cut metal must possess a tensile strength coefficient between 350 and 550 Rm N/mm², consisting advantageously of a stainless steel band or plate, preferably of ferritic stainless steel, and even more advantageously of a plate or band of ferritic 430 stainless steel, also called stainless 430 steel.

This method permits the fully automatic production of tweezer tips for ophthalmological microsurgery tweezers for cataract surgery, the latter presenting the desirable fineness (for example a width and thickness in the order of 0.25 mm in their active distal portion, and a prehension tooth of a length of 0.1 mm), sufficient rigidity to fulfill their function with the necessary efficacy and safety, and a tenderness resulting in blunting their prehension teeth at the end of the duration of an ocular microsurgery intervention such as a cataract operation. Furthermore, these tweezer teeth are made of a bio-compatible stainless metal, preferably ferritic stainless steel.

### BRIEF DESCRIPTION OF THE SEVERAL VIEWS OF THE DRAWINGS

The above aims, characteristics and advantages and still others will become clearer from the following description and the attached drawings.

FIG. 1 is an isometric perspective view of a production example of an ocular microsurgery tweezer with tips or tweezer teeth to the manufacture of which the method of the invention may be applied.

FIG. 2 is a front elevation view and at a larger scale, of a pair of tweezer tips shown in closed position.

FIG. 3 is a side elevation view (inside face) of one of the tweezer tips.

FIG. 4 is a front elevation view with partial section, of a tweezer tip.

FIG. 5 is a perspective view at a greatly enlarged scale, of the prehension micro-teeth that the tweezer tip ends are equipped with.

### DETAILED DESCRIPTION OF THE INVENTION

Reference to said drawings is made to describe an interesting, although by no means limiting, example of the application of the method and of execution of the tweezer teeth according to the invention.

The following is a description of a particularly advantageous application of the invention for the production of surgical tweezers, and, more specifically, of tweezers for ocular microsurgery.

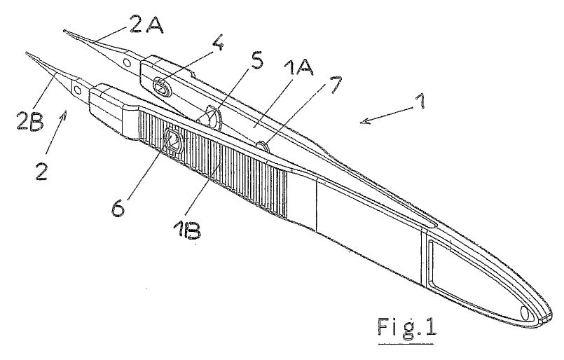

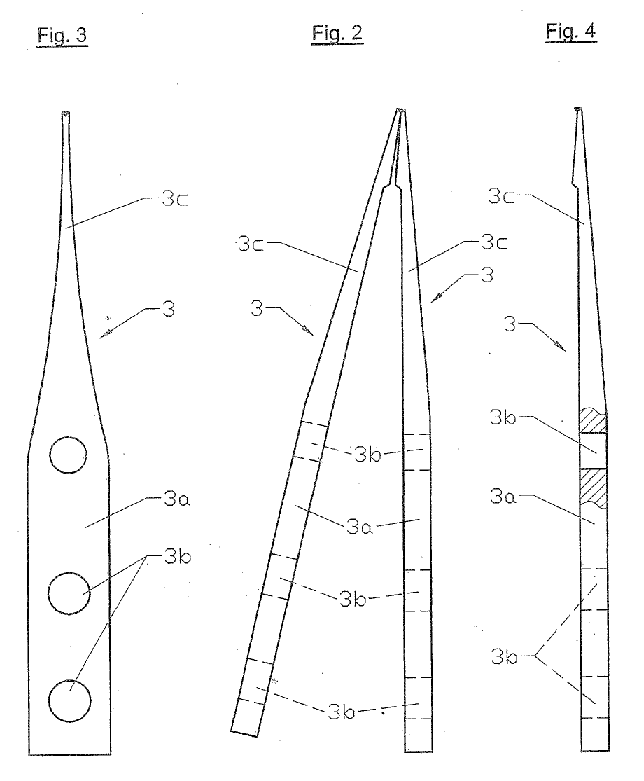

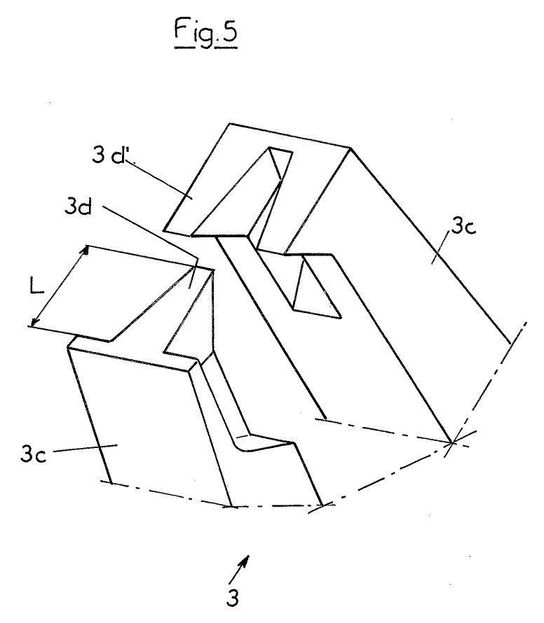

FIG. 1 shows an example of tweezers for ocular microsurgery with tweezer tips to the manufacture of which the method of the invention is applicable.

Such composite tweezers described in French Patent document FR-03/15172 include a prehension part 1 made of plastic material and formed by two branches or arms 1A, 1B, joined to each other at one of their ends to constitute the sleeve or the proximal part of the tweezer, and a tweezing part 2, constituted by metallic tips 3 implanted in the distal ends of said arms.

The tweezer tips 3 made are equipped, in a manner known per se, with a proximal part 3a which is meant to be anchored in the distal end of the arms of a tweezer, and a distal tweezing part 3c provided with a prehension tooth 3d or 3d′ (FIG. 5). This proximal part 3a is provided with holes 3b going through its thickness in order to contribute to the stability of this anchoring.

According to the method of the invention, the tweezer tips 3, provided with their prehension teeth 3d or 3d′, are die-cut and shaped under a cutting press equipped with appropriate die-cutting tools in a plate or band of stainless and bio-compatible metal. The die-cut metal must have a tensile strength coefficient between 350 and 550 Rm N/mm². This will advantageously be a stainless steel band or plate, preferably of ferritic stainless steel, and even more advantageously of a plate or band of ferritic 430 stainless steel, also called stainless 430 steel.

Interestingly, a band or plate of stainless steel will be used, for example of stainless 430 steel, with a thickness between 0.8 mm and 1 mm (0.8 mm and 1 mm included), for the die-cutting and the press-shaping of the tweezer tips 3.

The proximal part 3a of the prehension tips 3 presents a constant thickness and width over its entire length, for example a thickness of 1 mm and a width of 3 mm, whereas the distal part presents a section that becomes thinner towards its end, so as to presenting at its terminal part, being a very reduced section, for example in the order of 0.25×0.25 mm. On the other hand, the distal part 3c of each tweezer tip is provided with a prehension tooth 3d or 3d′ of a length L in the order of 0.1 mm and oriented in the direction of the prehension tooth 3d′ or 3d the distal part 3c of the other tweezer point 3 is provided with.

The cutting presses and the die-cutting tools that can be used for the execution of the method of the invention are known per se.

The combined utilization of ferritic stainless steel, particularly of the stainless 430 steel and the cutting press allow a high precision die-cutting and shaping of the tweezer teeth of tips.

It is to be emphasized here that ferritic stainless steel, and in particular the ferritic stainless 430 steel have until now not been used for the production of surgical instruments, because of its too high degree of malleability considered to be a deficiency which excludes it from the range of materials usable for the manufacture of such instruments (see Afnor Standards NF S94-090, April 2001: Material for surgical instruments—martensitic stainless steels hardened by precipitation, austenitic and austeno-ferritic steels).

According to the present invention, this preconception has been done away with and it has been considered that the relative malleability of certain stainless and bio-compatible metals such a ferritic 430 stainless steel was not prohibitory for its utilization in the manufacture of tweezer tips for microsurgery tweezers and in particular of tweezers for ophthalmological microsurgery intended for the manipulation of tissues or soft implants or very small dimensions (for example manipulation of crystalline, positioning of crystalline implants). In such applications, the tweezer tips, although they present a relatively high degree of malleability, also have a sufficient coefficient of rigidity or hardness to allow the manipulation of soft tissues with the indispensable efficacy and safety.

This method makes possible the industrial production of tips or teeth of composite precision tweezers, in particular of composite surgical tweezers, and consequently allows mass production of such instruments, in particular of disposable tweezers for ophthalmological microsurgery.

The invention also concerns composite precision tweezers and in particular composite surgical tweezers, and more specifically the tweezers for ocular or other microsurgery that are equipped with metallic tweezer teeth of tips of the kind that has been described before and result from the application of the method disclosed above.

1. Production method for tweezer tips for disposable ophthalmological microsurgery tweezers for cataract surgery, the method comprising the steps of:
molding a prehension part from a single piece of plastic material, wherein two arms joined to each other at one of end thereof to form a proximal part of the tweezer;
inserting metallic tips into distal ends of the arms to form a tweezing part, the tweezer tips being equipped with prehension teeth die-cut and shaped on a cutting press fitted with appropriate die-cutting tools, from a metal band or plate having a tensile strength coefficient between 350 and 550 Rm N/mm².

2. Method of production according to claim 1, wherein the tweezer tips are die-cut and shaped on a cutting press, from a stainless steel band or plate.

3. Method of production according to claim 1, wherein the tweezer tips are die-cut and shaped on a cutting press from a band or plate of ferritic stainless steel.

4. Method of production according to wherein the tweezer tips are die-cut and shaped on a cutting press from a band or plate of ferritic 430 steel.

5. Method of production according to claim 1, wherein the tweezer tips equipped with their prehension tooth are die-cut and shaped on a cutting press, from a band or plate presenting a thickness between 0.8 mm and 1 mm (0.8 mm and 1 mm included).

6. Disposable tweezer tips for tweezers for ophthalmological microsurgery, the tweezers each having arms, the tweezer tips comprising:
a proximal part anchored in a distal end of one arm of the tweezer and having a thickness in the order of 1 mm and a width in the order of 3 mm; and
a distal tweezing part with a section that becomes thinner in a direction of an end thereof, said end having a reduced section, in the order of 0.25×0.25 mm, a terminal part being provided with a prehension tooth of a length in the order of 0.1 mm, according to the method of claim 1.

7. Disposable ophthalmological microsurgery tweezers, the tweezers comprising:
a prehension part comprised of plastic material and formed by two arms joined to each other at one end thereof to form a proximal part of the tweezer; and,
a tweezing part being comprised of metallic tweezer tips implanted into distal ends of arms the tweezers being provided with tweezer tips according to claim 6.
